(19) United States
(12) Patent Application Publication
(10) Pub. No.: US 2026/0206813 A1
(43) Pub. Date: Jul. 23, 2026

(54) COMPOSITION FOR PROMOTING GHRELIN SECRETION

(71) Applicant: Meiji Co., Ltd., Tokyo (JP)

(72) Inventors: Kosaku OHINATA, Kyoto (JP); Kentaro KANEKO, Kyoto (JP); Eriko TANIGUCHI, Kyoto (JP); Hiroshi IWAKURA, Kyoto (JP); Yui FUNATSU, Tokyo (JP); Yoshitaka NAKAMURA, Tokyo (JP)

(73) Assignee: Meiji Co., Ltd., Tokyo (JP)

(21) Appl. No.: 19/135,996

(22) PCT Filed: Dec. 11, 2023
(86) PCT No.: PCT/JP2023/044210

§ 371 Date: Jun. 5, 2025

(30) Foreign Application Priority Data
Dec. 12, 2022 (JP) 2022-197732

(51) Int. Cl. A23L 33/115

(52) U.S. Cl. CPC A23L 33/115

(57) ABSTRACT

An object of the present invention is to provide a novel composition for use in promoting ghrelin secretion. According to the present invention, there is provided a composition for use in promoting ghrelin secretion, comprising, as an active ingredient(s), one or more substance(s) selected from the group consisting of an acylglycerol comprising palmitic acid and/or oleic acid as a constituent fatty acid(s), and oleic acid. The composition is preferably a composition for use in promoting appetite, ameliorating loss of appetite, promoting growth hormone secretion, improving malnutrition, promoting fat accumulation, increasing body weight, enhancing gastric acid secretion or enhancing gastric motility. The composition is preferably a food composition.

### CROSS-REFERENCE TO RELATED APPLICATION

The present application enjoys the benefit of priority from the prior Japanese Patent Application No. 2022-197732 filed on Dec. 12, 2022, the entire disclosure of which is incorporated herein by reference.

### TECHNICAL FIELD

The present invention relates to a composition for use in promoting ghrelin secretion.

### BACKGROUND ART

Ghrelin is a peptide hormone secreted primarily from the stomach and having an appetite enhancing action, and its activity is developed upon acylation modification of the third amino acid, serine residue, with octanoic acid. In addition to an eating enhancing action, ghrelin also has a fat accumulating action, a body weight increasing action, and the like, and is involved in regulation of energy metabolism. Ghrelin has also been reported to have a wide range of physiological actions, such as an action of promoting growth hormone secretion by acting on a growth hormone release promoting factor receptor in the pituitary gland, and an action of enhancing gastric acid secretion and gastric motility in the digestive system.

Promotion of ghrelin secretion is important for normal development of an appetite regulating function which is necessary for infants experiencing rapid physical and intellectual growth to take necessary energy, as well as for functional development due to physiological actions of ghrelin and growth hormone. On the other hand, the promotion of ghrelin secretion can also be expected to be effective in preventing and ameliorating loss of appetite and reduction in energy intake of aged persons, as well as reductions in muscle mass and muscle strength due to reduced secretion of growth hormone. In terms of clinical applications, clinical research is being conducted in expectation of effects for improving cachexia post gastrectomy and due to cancer, heart failure, chronic renal failure, autoimmune diseases, and the like, chronic obstructive pulmonary disease, functional gastrointestinal disorders, and anorexia nervosa.

Acylglycerols have a structure in which one to three fatty acids are ester-bound to the three hydroxyl groups of the glycerol backbone. A triacylglycerol accounts for the majority of lipids in foods, and is hydrolyzed with a lipase in the stomach and small intestine to produce a free fatty acid and a diacylglycerol or monoacylglycerol. The fatty acid bound to β-position (sn-2 position) of the glycerol backbone is hardly subject to the action of the lipase and is absorbed into small intestinal epithelial cells as 2-monoacylglycerol. Thus, various fatty acids and acylglycerols are present in the digestive tract, and each of the components is thought to have different influences on development of physiological activity and control of gene expression. Although it has been reported that free fatty acids suppress ghrelin secretion (Non-Patent Documents 1 and 2), the influence of acylglycerols on ghrelin secretion has not been elucidated.

### REFERENCE LIST

### Non-Patent Documents

Non-Patent Document 1: Z Gong et al., Am J Physiol Endocrinol Metab., 2014, 306 (1): E28-35
Non-Patent Document 2: X Lu et al., Am J Physiol Gastrointest Liver Physiol., 2012, 303 (3): G367-76

### SUMMARY OF THE INVENTION

An object of the present invention is to provide a novel composition for use in promoting ghrelin secretion.

The present inventors have now examined the influence of adding a free fatty acid and an acylglycerol to the mouse stomach-derived ghrelin-secreting cell line MGN3-1 on the secretion of active ghrelin. In particular, the present inventors have focused on differences in fatty acid composition and positional distribution on the glycerol backbone, and examined influences of various acylglycerols on the secretion of active ghrelin. As a result, the present inventors have found that an acylglycerol comprising oleic acid and palmitic acid as constituent fatty acids significantly promotes the secretion of active ghrelin. The present invention is based on these findings.

According to the present invention, the following inventions are provided.

[1] A composition for use in promoting ghrelin secretion and an agent for promoting ghrelin secretion, each comprising, as an active ingredient(s), one or more substance(s) selected from the group consisting of an acylglycerol comprising palmitic acid and/or oleic acid as a constituent fatty acid(s), and oleic acid.

[2] The composition and agent according to [1], wherein, in the acylglycerol, the palmitic acid and/or the oleic acid are/is bound to sn-1 position and/or sn-2 position of glycerol.

[3] The composition and agent according to [1], wherein the acylglycerol comprises the palmitic acid and the oleic acid as the constituent fatty acids.

[4] The composition and agent according to [2], wherein, in the acylglycerol, an optional fatty acid is bound to sn-3 position of the acylglycerol. [5] The composition and agent according to [4], wherein the optional fatty acid is palmitic acid or oleic acid.

[6] The composition and agent according to [1], wherein the acylglycerol is one or more selected from the group consisting of 1-monopalmitin, 2-monopalmitin, 1-monoolein, 2-monoolein, 1-oleoyl-2-palmitoylglycerol, 1-palmitoyl-2-oleoylglycerol, 1,3-dioleoyl-2-palmitoylglycerol, and 1,3-dipalmitoyl-2-oleoylglycerol.

[7] The composition and agent according to [1], wherein the amount of the active ingredient(s) in the composition is 1 to 95% by mass.

[8] The composition and agent according to [1], which is for use in promoting appetite, ameliorating loss of appetite, promoting growth hormone secretion, improving malnutrition, promoting fat accumulation, increasing body weight, enhancing gastric acid secretion or enhancing gastric motility.

[9] The composition and agent according to [1], which is a food composition.

[10] The composition and agent according to [1], which is a food composition for infants and/or aged persons.

[11] A method for promoting ghrelin secretion, a method for promoting or enhancing appetite, a method for ameliorating or alleviating loss of appetite, a method for promoting or enhancing growth hormone secretion, a method for improving or alleviating malnutrition, a method for promoting or enhancing fat accumulation, a method for increasing body weight, a method for promoting or enhancing gastric acid secretion, or a method for promoting or enhancing gastric motility, the method comprising feeding or administering, to a subject in need thereof, one or more substance(s) selected from the group consisting of an acylglycerol comprising palmitic acid and/or oleic acid as a constituent fatty acid(s) and oleic acid, or a composition comprising the one or more substance(s).

[12] Use of one or more substance(s) selected from the group consisting of an acylglycerol comprising palmitic acid and/or oleic acid as a constituent fatty acid(s) and oleic acid, or a composition comprising the one or more substance(s), for manufacture of an agent for promoting ghrelin secretion, an agent for promoting or enhancing appetite, an agent for ameliorating or alleviating loss of appetite, an agent for promoting or enhancing growth hormone secretion, an agent for improving or alleviating malnutrition, an agent for promoting or enhancing fat accumulation, an agent for increasing body weight, an agent for promoting or enhancing gastric acid secretion, or an agent for promoting or enhancing gastric motility; as an agent for promoting ghrelin secretion, an agent for promoting or enhancing appetite, an agent for ameliorating or alleviating loss of appetite, an agent for promoting or enhancing growth hormone secretion, an agent for improving or alleviating malnutrition, an agent for promoting or enhancing fat accumulation, an agent for increasing body weight, an agent for promoting or enhancing gastric acid secretion, or an agent for promoting or enhancing gastric motility; or in a method for promoting ghrelin secretion, a method for promoting or enhancing appetite, a method for ameliorating or alleviating loss of appetite, a method for promoting or enhancing growth hormone secretion, a method for improving or alleviating malnutrition, a method for promoting or enhancing fat accumulation, a method for increasing body weight, a method for promoting or enhancing gastric acid secretion, or a method for promoting or enhancing gastric motility.

[13] One or more substance(s) selected from the group consisting of an acylglycerol comprising palmitic acid and/or oleic acid as a constituent fatty acid(s) and oleic acid, or a composition comprising the one or more substance(s), for use in promoting ghrelin secretion, promoting or enhancing appetite, ameliorating or alleviating loss of appetite, promoting or enhancing growth hormone secretion, improving or alleviating malnutrition, promoting or enhancing fat accumulation, increasing body weight, promoting or enhancing gastric acid secretion, or promoting or enhancing gastric motility.

In the present specification, the composition of [1] above is sometimes referred to as “the composition of the present invention.” Also, in the present specification, the agent of [1] above is sometimes referred to as “the agent of the present invention.”

The active ingredient of the composition and agent of the present invention is an oil and/or fat that has been used as a raw material for foods for many years. Therefore, the composition and agent of the present invention are advantageous in providing a ghrelin secretion promoting effect, and being highly safe without any concern about side effects even when taken continuously over a long period of time.

### BRIEF DESCRIPTION OF THE DRAWINGS

FIG. 1 is a view showing the ghrelin secretion activity of 2-monopalmitin (30 μM) (n=4) (Example 1). Measured values are indicated as mean±standard error, and * indicates p<0.05.

FIG. 2 is a view showing the ghrelin secretion activity of 2-monopalmitin, 1-monoolein or 2-monoolein (each 30 μM) (n=4) (Example 1). Measured values are indicated as mean±standard error, and lowercase alphabetic characters indicate significant differences between different characters (p<0.05).

### DETAILED DESCRIPTION OF THE INVENTION

The composition and agent of the present invention each comprise, as an active ingredient(s), one or more substance(s) selected from the group consisting of an acylglycerol comprising palmitic acid and/or oleic acid, and oleic acid. In the present invention, the active ingredient(s) “one or more substance(s) selected from the group consisting of an acylglycerol comprising palmitic acid and/or oleic acid as a constituent fatty acid(s), and oleic acid” is/are sometimes referred to as “the acylglycerol and/or oleic acid of the present invention.” In the present invention, the “acylglycerol comprising palmitic acid and/or oleic acid as a constituent fatty acid(s)” is a substance in which palmitic acid and/or oleic acid are/is ester-bound to glycerol, and refers to all compounds that are identical in terms of chemical formula. In the present invention, the active ingredient “acylglycerol comprising palmitic acid and/or oleic acid as a constituent fatty acid(s)” is sometimes referred to as “the acylglycerol of the present invention.” In the present invention, the active ingredient oleic acid is sometimes referred to as “the oleic acid of the present invention.”

The acylglycerol of the present invention includes an acylglycerol in which palmitic acid is ester-bound to sn-2 position of glycerol, an acylglycerol in which oleic acid is ester-bound to the sn-2 position of the glycerol, an acylglycerol in which palmitic acid is ester-bound to sn-1 position of the glycerol, and an acylglycerol in which oleic acid is ester-bound to the sn-1 position of the glycerol. The acylglycerol of the present invention also includes monoacylglycerols, diacylglycerols and triacylglycerols. That is, the above-described acylglycerols specified in fatty acid and its positional distribution may include either no fatty acids or optional fatty acids (which may be identical or different) present at the two positions other than the position where the presence of the fatty acid is specified, or, alternatively, may include an optional fatty acid present at one of the two positions other than the position where the presence of the fatty acid is specified, and no fatty acid present at the other position. For example, the acylglycerol in which palmitic acid or oleic acid is ester-bound to the sn-2 position of the glycerol may include either no fatty acids or optional fatty acids (which may be identical or different) present at the sn-1 position and the sn-3 position, or, alternatively, may include an optional fatty acid present at one of the sn-1 position and the sn-3 position and no fatty acid present at the other position.

The optional fatty acid constituting the acylglycerol includes saturated fatty acids and unsaturated fatty acids having 2 to 24 carbon atoms, such as oleic acid, palmitic acid, caprylic acid, and docosahexaenoic acid.

The acylglycerol of the present invention is preferably an acylglycerol comprising either palmitic acid or oleic acid or both palmitic acid and oleic acid as a constituent fatty acid(s). Preferred examples of the acylglycerol of the present invention include the following (1) to (4):
(1) an acylglycerol in which palmitic acid or oleic acid is ester-bound to the sn-2 position of the glycerol and no fatty acid is present at the sn-1 position or the sn-3 position;
(2) an acylglycerol in which palmitic acid or oleic acid is ester-bound to the sn-2 position of the glycerol and palmitic acid or oleic acid is present at either the sn-1 position or the sn-3 position or both the sn-1 position and the sn-3 position;
(3) an acylglycerol in which palmitic acid or oleic acid is ester-bound to the sn-1 position of the glycerol and no fatty acid is present at the sn-2 position or the sn-3 position; and
(4) an acylglycerol in which palmitic acid or oleic acid is ester-bound to the sn-1 position of the glycerol and palmitic acid or oleic acid is present at either the sn-2 position or the sn-3 position or both the sn-2 position and the sn-3 position. In the acylglycerols (2) and (4) among the acylglycerols indicated above, either no fatty acid or an optional fatty acid may be present at the position where the presence of palmitic acid and oleic acid is not specified, and preferably no fatty acid is present at the position.

Particularly preferred examples of the acylglycerol of the present invention include 1-monopalmitin, 2-monopalmitin, 1-monoolein, 2-monoolein, 1-oleoyl-2-palmitoylglycerol (OP), 1-palmitoyl-2-oleoylglycerol (PO), 1,3-dioleoyl-2-palmitoylglycerol (OPO), and 1,3-dipalmitoyl-2-oleoylglycerol (POP).

The acylglycerol of the present invention may be either a pure product or a mixture with another substance. For example, the acylglycerol of the present invention may be measured by a known measurement method, and the raw material of which the presence has been confirmed may be used as it is.

The oleic acid of the present invention may be either a pure product or a mixture with another substance. For example, the oleic acid of the present invention may be measured by a known measurement method, and the raw material of which the presence has been confirmed may be used as it is. The oleic acid of the present invention may also be oleic acid liberated upon hydrolysis of an acylglycerol with a lipase. The lipase which hydrolyzes an acylglycerol is, for example, triacylglycerol lipase (EC 3.1.1.3, sometimes referred to as a gastric lipase in the present specification). The acylglycerol from which oleic acid is liberated by the lipase can be an acylglycerol comprising oleic acid as a constituent fatty acid, and can preferably be an acylglycerol in which oleic acid is bound to the sn-3 position of the glycerol, such as 1,3-dioleoyl-2-palmitoylglycerol (OPO) or 1-linoleyl-2-palmitoyl-3-oleoylglycerol.

According to an aspect of the composition and agent of the present invention, there are provided a composition and an agent each comprising the acylglycerol and/or oleic acid of the present invention as an active ingredient(s). A representative raw material (mixture) rich in the acylglycerol of the present invention is lard, and representative raw materials (mixtures) rich in the oleic acid of the present invention are olive oil, high oleic sunflower oil, and high oleic safflower oil. These materials can also be used in the composition and agent of the present invention. Alternatively, a commercially available product of the acylglycerol of the present invention and a commercially available product of the oleic acid of the present invention may be purchased and appropriately blended with another raw material for use in the composition and agent of the present invention.

The composition and agent of the present invention may each comprise the acylglycerol and/or oleic acid of the present invention alone, or may each comprise the acylglycerol and/or oleic acid of the present invention in admixture with another component. The lower limit value of the amount of the active ingredient(s) of the present invention (the acylglycerol and/or oleic acid of the present invention) in the composition and agent of the present invention can be 1% by mass or 2% by mass, preferably 3% by mass, and more preferably 5% by mass. The upper limit value of the amount can be 95% by mass or 93% by mass, preferably 30% by mass or 25% by mass, and more preferably 10% by mass or 5% by mass. These lower and upper limit values can be combined arbitrarily, and the range of the amount can be, for example, 1 to 95% by mass, 2 to 95% by mass, 2 to 30% by mass, 5 to 30% by mass, or 2 to 5% by mass. The lower limit value of the amount of the acylglycerol of the present invention in the composition and agent of the present invention can be 1% by mass or 2% by mass, preferably 3% by mass, and more preferably 5% by mass. The upper limit value of the amount can be 95% by mass or 93% by mass, preferably 30% by mass or 25% by mass, and more preferably 10% by mass or 5% by mass. These lower and upper limit values can be combined arbitrarily, and the range of the amount can be, for example, 1 to 95% by mass, 2 to 95% by mass, 2 to 30% by mass, 5 to 30% by mass, or 2 to 5% by mass. The lower limit value of the amount of the oleic acid of the present invention in the composition and agent of the present invention can be 1% by mass or 2% by mass, preferably 3% by mass, and more preferably 5% by mass. The upper limit value of the amount can be 95% by mass or 93% by mass, preferably 30% by mass or 25% by mass, and more preferably 10% by mass or 5% by mass. These lower and upper limit values can be combined arbitrarily, and the range of the amount can be, for example, 1 to 95% by mass, 2 to 95% by mass, 2 to 30% by mass, 5 to 30% by mass, or 2 to 5% by mass. In the present invention, the agent of the present invention can consist of the acylglycerol and/or oleic acid of the present invention, and the composition of the present invention can comprise the acylglycerol and/or oleic acid of the present invention.

The amount of the active ingredient(s) of the present invention in the composition and agent of the present invention can also be determined based on the mass of solids. That is, the lower limit value of the amount of the active ingredient(s) of the present invention (the acylglycerol and/or oleic acid of the present invention) in the entire solids of the composition and agent of the present invention can be 1% by mass or 2% by mass, preferably 3% by mass or 5% by mass, and more preferably 10% by mass or 15% by mass. The upper limit value of the amount can be 95% by mass or 93% by mass, preferably 50% by mass or 40% by mass, and more preferably 30% by mass or 25% by mass. These lower and upper limit values can be combined arbitrarily, and the range of the amount can be, for example, 1 to 95% by mass, 2 to 95% by mass, 2 to 30% by mass, or 5 to 30% by mass. The lower limit value of the amount of the acylglycerol in the entire solids of the composition and agent of the present invention can be 1% by mass or 2% by mass, preferably 3% by mass or 5% by mass, and more preferably 10% by mass or 15% by mass. The upper limit value of the amount can be 95% by mass or 93% by mass, preferably 50% by mass or 40% by mass, and more preferably 30% by mass or 25% by mass. These lower and upper limit values can be combined arbitrarily, and the range of the amount can be, for example, 1 to 95% by mass, 2 to 95% by mass, 2 to 30% by mass, or 5 to 30% by mass. The lower limit value of the amount of the oleic acid in the entire solids of the composition and agent of the present invention can be 1% by mass or 2% by mass, preferably 3% by mass or 5% by mass, and more preferably 10% by mass or 15% by mass. The upper limit value of the amount can be 95% by mass or 93% by mass, preferably 50% by mass or 40% by mass, and more preferably 30% by mass or 25% by mass. These lower and upper limit values can be combined arbitrarily, and the range of the amount can be, for example, 1 to 95% by mass, 2 to 95% by mass, 2 to 30% by mass, or 5 to 30% by mass. In the present specification, the term “solids” refers to components except moisture.

The composition and agent of the present invention may each comprise an oil and/or fat such as an oil and/or fat containing a medium-chain fatty acid (e.g., a medium-chain fatty acid glyceride) or a general edible oil and/or fat (e.g., an oil and/or fat mainly containing a long-chain fatty acid, such as soybean oil), in addition to the acylglycerol and/or oleic acid of the present invention. The amount of the oil and/or fat other than the acylglycerol and/or oleic acid of the present invention in the composition and agent of the present invention can be, for example, 0 to 99% by mass, and preferably 0 to 70% by mass. The amount of the oil and/or fat other than the acylglycerol and/or oleic acid of the present invention in the entire solids of the composition and agent of the present invention can be, for example, 0 to 99% by mass, and preferably 0 to 70% by mass.

The composition and agent of the present invention can be used to promote ghrelin secretion. Here, the meaning of the “promotion of secretion,” as used herein, includes not only promoting ghrelin secretion, but also suppressing a decrease in ghrelin secretion and maintaining ghrelin secretion. Furthermore, in the present invention, the promotion of ghrelin secretion can be promotion of ghrelin secretion in the stomach, and is preferably promotion of active ghrelin (acylated ghrelin) in the stomach of humans or non-human animals (e.g., mammals other than humans).

In the present invention, the promotion of ghrelin secretion can be evaluated using a secretion promotion rate as an index. Specifically, the ratio (%) of the amount of ghrelin secreted in the presence of the composition and agent of the present invention to the amount of ghrelin secreted in the absence of the composition and agent of the present invention can be defined as the secretion promotion rate. A secretion promotion amount can be evaluated using, for example, the amount of increase of ghrelin in cell culture solution or the amount of increase of ghrelin levels in the blood in a living body as an index. In the present invention, it can be determined that there is a ghrelin secretion promoting effect when this secretion promotion rate (%) exceeds 110%, 120%, 130%, 150%, 170% or 200%, or when the amount of ghrelin secreted in the presence of the composition and agent of the present invention is statistically significantly increased as compared with the amount of ghrelin secreted in the absence of the composition and agent of the present invention.

According to the Examples which will be described below, the acylglycerol and oleic acid of the present invention can promote ghrelin secretion. It is known that the promotion of ghrelin secretion results in promoting appetite, ameliorating loss of appetite, promoting growth hormone secretion, improving malnutrition, promoting fat accumulation, increasing body weight, enhancing gastric acid secretion or enhancing gastric motility (Mercedes Gil-Campos, et al., Br J Nutr. 2006, 96 (2): 201-26, Masayasu Kojima, et al., Nature. 1999, 402 (6762): 656-60, Yutaka Masuda, et al., Biochem Biophys Res Commun., 2000, 276 (3): 905-8). That is, these effects can be expected by feeding or administering the acylglycerol and/or oleic acid of the present invention. Hence, the composition and agent of the present invention can be fed or administered to a subject with low ghrelin secretion, a subject who desires to enjoy the effects obtained by promoting ghrelin secretion, or a subject for whom the promotion of ghrelin secretion is desirable. Examples of such subjects include infants and aged persons. The composition and agent of the present invention have an appetite increasing action and a growth hormone secretion promoting action through ghrelin secretion, and are therefore suitable for promoting normal growth and development of infants and for improving the nutritional state of aged persons who have fallen into a malnutrition state. The target for feeding or administration of the composition and agent of the present invention include healthy persons.

In the present invention, the amount of ghrelin secreted in the subject can be measured before the composition and agent of the present invention are fed or administered to the subject. The amount of ghrelin secreted in the target for feeding or administration can be specified using the concentration of ghrelin in the blood (e.g., serum or plasma) as an index. For example, the composition and agent of the present invention can be fed or administered to a subject whose concentration of ghrelin in the plasma is 10 pg/ml or less, 20 pg/ml or less, or 30 pg/ml or less. The concentration of ghrelin in the plasma can be measured by an immunological measurement method such as the ELISA method.

The acylglycerol and oleic acid of the present invention can each be used for promoting or enhancing appetite, ameliorating or alleviating loss of appetite, promoting or enhancing growth hormone secretion, improving or alleviating malnutrition, promoting or enhancing fat accumulation, increasing body weight, promoting or enhancing gastric acid secretion, or promoting or enhancing gastric motility. That is, according to the present invention, there is provided an agent for promoting or enhancing appetite, an agent for ameliorating or alleviating loss of appetite, an agent for promoting or enhancing growth hormone secretion, an agent for improving or alleviating malnutrition, an agent for promoting or enhancing fat accumulation, an agent for increasing body weight, an agent for promoting or enhancing gastric acid secretion, or an agent for promoting or enhancing gastric motility, the agent comprising the acylglycerol and/or oleic acid as an active ingredient(s).

The composition and agent of the present invention can be provided in the form of medicaments and quasi-drugs (e.g., pharmaceutical compositions), foods (e.g., food compositions), feeds (e.g., feed compositions), and the like, and can be implemented according to the following descriptions.

The acylglycerol and oleic acid of the present invention can be administered orally to humans and non-human animals. Oral agents include granules, powders, tablets (including sugar-coated tablets), pills, capsule agents, syrup agents, emulsions, and suspensions. These formulations can be formulated using a pharmaceutically acceptable carrier by a technique commonly used in the art. The pharmaceutically acceptable carrier includes excipients, binders, diluents, additives, perfumes, buffers, thickeners, colorants, stabilizers, emulsifiers, dispersants, suspending agents, preservatives, and the like.

The acylglycerol and oleic acid of the present invention can also be administered into bodies of humans and non-human animals by a method other than oral administration, such as tube administration or nasal tube administration, depending on the forms of the composition and agent of the present invention. For example, the composition and agent of the present invention are made into viscous liquid compositions comprising the acylglycerol and oleic acid of the present invention, or semi-solid compositions comprising the acylglycerol and oleic acid of the present invention, whereby they can be fed or administered to humans and non-human animals whose chewing and swallowing functions have been impaired and to whom/which the composition and agent of the present invention cannot therefore be fed or administered orally. The composition and agent of the present invention are fed or administered by a method other than oral feeding, whereby they can be expected to exert the ghrelin secretion promoting action in the subject, even if the chewing and swallowing functions of the target for feeding or administration are underdeveloped due to young age or have been impaired due to aging or the like.

The acylglycerol and oleic acid of the present invention can be fed orally to humans and non-human animals. When the acylglycerol and oleic acid of the present invention are fed orally, they may be in an isolated, purified or partially purified form, or in the form of a food or raw material for foods comprising the acylglycerol and oleic acid of the present invention.

When the acylglycerol and oleic acid of the present invention are provided as foods, they can be contained in a food, and such a food is a food containing effective amounts of the acylglycerol and oleic acid of the present invention. In the present invention, when the acylglycerol and oleic acid of the present invention are provided as foods, a food or raw material already containing them can be provided as the food of the present invention. Such a food is a food containing effective amounts of the acylglycerol and oleic acid of the present invention. Here, the phrase “containing effective amounts” of the acylglycerol and oleic acid of the present invention, as used herein, refers to amounts of the acylglycerol and oleic acid of the present invention to be taken within ranges as will be described later, when individual foods are fed in a normally-eaten amount. The meaning of the “food,” as used herein, includes health foods, functional foods, foods with health claims (such as foods for specified health uses, foods with nutrient function claims, and foods labeled with functions), foods for special dietary uses (such as foods for persons with difficulty in swallowing, infant formula, milk powder for expectant and nursing mothers, and foods for sick persons), nutritional supplements (supplements), and foods for infants.

The form of the “food” is not particularly limited, and may be, for example, a liquid form such as a beverage or a liquid diet; a paste, semi-liquid, or gel form; or a solid, bar, or powder form. When the composition and agent of the present invention are used in the form of powder, they can be produced by using a means such as spray drying or freeze drying.

When the composition and agent of the present invention are provided as foods containing the acylglycerol and/or oleic acid of the present invention, the foods can be produced according to a normal food production method, except that the acylglycerol and/or oleic acid of the present invention are/is blended. That is, the food of the present invention can be prepared by adding the acylglycerol and/or oleic acid of the present invention to various foods (e.g., cow milk, soft drinks, fermented milk, yogurt, chocolate, gummies, cheese, bread, biscuits, cookies, crackers, pizza crust, jelly, ice cream, high-energy supplements, high-energy pastes, prepared milk powder, liquid diets, foods for special dietary uses, foods for sick persons, comprehensive nutritional foods, nutritional supplements, frozen foods, processed foods, and other commercially available foods) or their raw materials, regardless of whether the foods are in a liquid, solid, powder, or any other form. Furthermore, the acylglycerol and oleic acid of the present invention in various forms (liquid, solid, powder, paste, or any other form) can be added to water, foods and beverages, or meals and taken by the subject himself/herself. Especially, in the present invention, effective amounts of the acylglycerol and oleic acid of the present invention are blended into a liquid diet, whereby a food having also a ghrelin secretion promoting function can be prepared. Such a food is advantageous because it can be fed or administered to infants whose chewing and swallowing functions are underdeveloped, or to sick persons and aged persons whose chewing and swallowing functions have been impaired.

When the composition and agent of the present invention are provided in the form of a food or raw material (especially, a processed raw material) already containing the acylglycerol and/or oleic acid of the present invention, examples of such foods and raw materials include edible oils and/or fats such as lard and vegetable oils and/or fats (olive oil, high oleic sunflower oil, and high oleic safflower oil). The composition and agent of the present invention are suitable for feeding to infants or middle-aged and older persons and aged persons who require the ghrelin secretion promoting action.

In the present invention, for example, the acylglycerol and oleic acid of the present invention can be combined with another raw material and the like and provided as a composition or supplement aimed at promoting ghrelin secretion.

The daily intake amount or dose of the composition and agent of the present invention as medicaments or foods varies depending on the pathology, age, symptoms and body weight of the subject, the intended use, and the like, and thus is not particularly limited. When the acylglycerol and oleic acid of the present invention are fed or administered for the purpose of promoting ghrelin secretion, the daily intake amount or dose (in terms of solids) for adults of the acylglycerol and oleic acid of the present invention is not particularly limited, but the lower limit thereof is, for example, 0.01 g, and preferably 0.1 g. The upper limit is, for example, 65 g or 60 g, and preferably 5 g or 1 g. These lower and upper limit values can be combined arbitrarily. Furthermore, the number of times and frequency of feeding or administration can be appropriately determined depending on the required level of ghrelin secretion promoting action. The intake amount and dose as well as feeding interval and administration interval can be determined by the subject's attending physician, pharmacist, registered dietitian, dietitian, care worker, care manager, helper, staff at a care facility, caregiver such as the subject's family member, or the subject himself/herself.

The composition and agent of the present invention is used in combination with another composition and agent that can be fed orally, without any limitation. For example, the ghrelin secretion promoting action/effect can be further enhanced by using the composition and agent of the present invention with materials and compositions that can be expected to exert the ghrelin secretion promoting action, such as fermented milk and lactic acid bacteria.

The composition and agent of the present invention can be provided in a daily intake amount which is effective for the ghrelin secretion promoting action. In this case, the composition and agent of the present invention may be packaged so that effective daily intake amounts of the acylglycerol and oleic acid of the present invention can be taken, and the package form may be a single package or multiple packages, as long as the effective daily intake amounts can be taken. When the composition and agent of the present invention are provided in a package form, it is desirable that the package should give a description about the intake amounts so that the effective daily intake amounts can be taken, or that a document with the description should be provided together. In addition, when the effective daily intake amounts are provided in multiple packages, the multiple packages for the effective daily intake amounts can be provided in a set, for convenience of feeding.

The package form for providing the composition and agent of the present invention is not particularly limited as long as it defines a certain amount, and examples thereof include wrapping paper, bags, soft bags, paper containers, cans, bottles, capsules, and other containers that can accommodate the composition and agent of the present invention.

In order to exert the effect, the composition and agent of the present invention are desirably administered or fed continuously for at least one week, and the period of administration or feeding is preferably continuously two weeks or more (e.g., 2 to 14 weeks), and more preferably continuously three weeks or more (e.g., 3 to 21 weeks). The term “continuously,” as used herein, means continuing administration or feeding at least once (e.g., once to seven times) per week. When the composition and agent of the present invention are provided in a packaged form, effective intake amounts thereof for a certain period of time (for example, one week) may be provided in a set, for the purpose of continuous feeding.

The food of the present invention may be labeled with an indication that it has an action of promoting ghrelin secretion, an action of promoting or enhancing appetite, an action of ameliorating or alleviating loss of appetite, an action of promoting or enhancing growth hormone secretion, an action of improving or alleviating malnutrition, an action of promoting or enhancing fat accumulation, an action of increasing body weight, promoting or enhancing gastric acid secretion, or an action of promoting or enhancing gastric motility. In this case, in order to make the indication easy for consumers to understand, the food of the present invention may be labeled with, for example, some or all of the following indications.
The food is helpful in increasing appetite which reduces due to aging.
The food is helpful in ameliorating loss of appetite due to stress or feeling of fatigue.
Ghrelin works to promote growth hormone secretion which reduces due to aging. Growth hormone is known to have effects for promoting protein synthesis and tissue repair and regeneration, recovering from fatigue, and the like.
The food is helpful in improving a malnutrition state which is prone to occur due to aging.
The food is helpful in improving a malnutrition state due to stress or feeling of fatigue.
The food works to promote lipid accumulation which is helpful in replenishing energy liable to become insufficient due to aging.
The food works to promote lipid accumulation which is helpful in replenishing energy liable to become insufficient due to stress or feeling of fatigue.
The food is helpful in maintaining and increasing body weight which reduces due to aging.
The food is helpful in maintaining and increasing body weight which reduces due to stress or feeling of fatigue.
The food improves the capability to secrete gastric acid which reduces due to aging, and is helpful in promoting digestion.
The food improves gastric motility which reduces due to aging, and is helpful in promoting digestion.

According to another aspect of the present invention, there is provided a method for promoting ghrelin secretion, a method for promoting or enhancing appetite, a method for ameliorating or alleviating loss of appetite, a method for promoting or enhancing growth hormone secretion, a method for improving or alleviating malnutrition, a method for promoting or enhancing fat accumulation, a method for increasing body weight, a method for promoting or enhancing gastric acid secretion, or a method for promoting or enhancing gastric motility, the method comprising feeding or administering, to a subject in need thereof, effective amounts of the acylglycerol and oleic acid of the present invention or a composition comprising them. The method of the present invention can be implemented according to the descriptions about the composition and agent of the present invention.

According to another aspect of the present invention, there is provided use of the acylglycerol and oleic acid of the present invention or a composition comprising them, for manufacture of an agent for promoting ghrelin secretion, an agent for promoting or enhancing appetite, an agent for ameliorating or alleviating loss of appetite, an agent for promoting or enhancing growth hormone secretion, an agent for improving or alleviating malnutrition, an agent for promoting or enhancing fat accumulation, an agent for increasing body weight, an agent for promoting or enhancing gastric acid secretion, or an agent for promoting or enhancing gastric motility; as an agent for promoting ghrelin secretion, an agent for promoting or enhancing appetite, an agent for ameliorating or alleviating loss of appetite, an agent for promoting or enhancing growth hormone secretion, an agent for improving or alleviating malnutrition, an agent for promoting or enhancing fat accumulation, an agent for increasing body weight, an agent for promoting or enhancing gastric acid secretion, or an agent for promoting or enhancing gastric motility; or in a method for promoting ghrelin secretion, a method for promoting or enhancing appetite, a method for ameliorating or alleviating loss of appetite, a method for promoting or enhancing growth hormone secretion, a method for improving or alleviating malnutrition, a method for promoting or enhancing fat accumulation, a method for increasing body weight, a method for promoting or enhancing gastric acid secretion, or a method for promoting or enhancing gastric motility. The use of the present invention can be implemented according to the descriptions about the composition and agent of the present invention and the method of the present invention.

According to still another aspect of the present invention, there are/is provided the acylglycerol and oleic acid of the present invention or a composition comprising them, for use in promoting ghrelin secretion, promoting or enhancing appetite, ameliorating or alleviating loss of appetite, promoting or enhancing growth hormone secretion, improving or alleviating malnutrition, promoting or enhancing fat accumulation, increasing body weight, promoting or enhancing gastric acid secretion, or promoting or enhancing gastric motility. The invention directed to these substances can be implemented according to the descriptions about the composition and agent of the present invention and the method of the present invention.

The method of the present invention and the use of the present invention may be use in mammals including humans, and are intended to involve both therapeutic use and non-therapeutic use. The term “non-therapeutic,” as used herein, means elimination of operating, treating or diagnosing activities to a human (i.e., medical activities to a human), and specifically means elimination of a method of performing operation or treatment of, or diagnosis involving, a human by a doctor or a person who receives an instruction from the doctor.

### EXAMPLES

The present invention will be described in more detail by way of the following examples, but is not limited to these examples.

### Example 1: Influence of Acylglycerol and Fatty Acid on Secretion of Active Ghrelin

### (1) Testing Method

The mouse stomach-derived ghrelin-secreting cell line MGN3-1 was seeded at 1×10⁵ cells/well in a 96-well plate and cultured for 24 hours. After culturing, the cells were washed with 200 μL of Dulbecco's PBS, and 100 μL of each solution containing one test substance as indicated in Table 1 (buffer solution: 50 μM sodium octanoate/DMEM) was added (test group). The concentration of the test substance was applied at 10 μM or 30 μM. In the control group, 100 μL of a buffer solution without test substance was added. The number of trials performed, n, for each group was 4. After each group was cultured for 2 hours, a culture solution was recovered and centrifuged (800 g, 5 minutes, 4° C.) to obtain 80 μL of a supernatant. The supernatant was added with 8 μL of 1 N HCl and stored at −80° C. Next, active ghrelin (acylated ghrelin) in the sample was measured by the ELISA method (Ghrelin (Acylated) EIA Kit A05117, Bertin Pharma). Average values of measured values of the concentrations of ghrelin secreted when the test substances as indicated in Table 2 or Table 3 were added were calculated and indicated as values (%) assuming that the value of the control is 100 (Tables 2 and 3). The test was conducted again using the same testing method as described above, and the concentration of active ghrelin (acylated ghrelin) secreted in the sample was measured (FIGS. 2 and 3).

TABLE 1
Test substance name and CAS registration number

| Test substance | | CAS registration |
|---|---|---|
| No. | Substance name | number |
| 1 | Palmitic acid | 57-10-3 |
| 2 | Oleic acid | 112-80-1 |
| 3 | 1-Monopalmitin | 542-44-9 |
| 4 | 2-Monopalmitin | 23470-00-0 |
| 5 | 1-Monoolein | 111-03-5 |
| 6 | 2-Monoolein | 3443-84-3 |
| 7 | 1-Oleoyl-2-palmitoylglycerol (OP) | 3331-34-8 |
| 8 | 1-Palmitoyl-2-oleoylglycerol (PO) | 3123-73-7 |
| 9 | 1,3-Dioleoyl-2-palmitoylglycerol (OPO) | 1716-07-0 |
| 10 | 1,3-Dipalmitoyl-2-oleoylglycerol (POP) | 2190-25-2 |

### (2) Statistical Analysis

Statistical analysis was performed using a t-test or one-way ANOVA (one-way analysis of variance), and a multiple comparison test was conducted using the Tukey-Kramer test.

### (3) Result

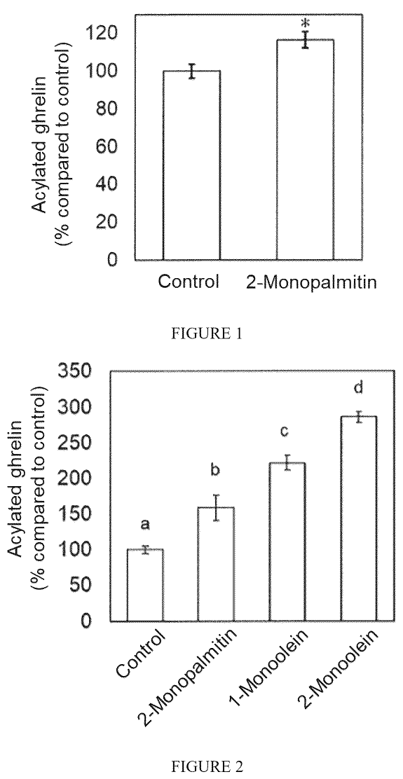

The results were shown in Tables 2 and 3 and FIGS. 1 and 2.

TABLE 2
Acylated ghrelin level (% control) induced
by acylglycerol and fatty acids

| Test substance | | |
|---|---|---|
| No. | Substance name | Acylated ghrelin (% control) |
| Control | Not added | 100 |
| 1 | Palmitic acid (10 μM) | 82 |
| | Palmitic acid (30 μM) | 107 |
| 2 | Oleic acid (10 μM) | 146 |
| | Oleic acid (30 μM) | 138 |
| 3 | 1-Monopalmitin (10 μM) | 105 |
| | 1-Monopalmitin (30 μM) | 134 |
| 4 | 2-Monopalmitin (10 μM) | 168 |
| | 2-Monopalmitin (30 μM) | 218 |
| 5 | 1-Monoolein (10 μM) | 177 |
| | 1-Monoolein (30 μM) | 209 |
| 6 | 2-Monoolein (10 μM) | 193 |
| | 2-Monoolein (30 μM) | 206 |

TABLE 3
Acylated ghrelin levels (% control) induced
by acylglycerols and fatty acids at 30 μM

| Test substance | | Acylated ghrelin |
|---|---|---|
| No. | Substance name | (% control) |
| Control | Not added | 100 |
| 2 | Oleic acid | 221 |
| 4 | 2-Monopalmitin | 389 |
| 5 | 1-Monoolein | 388 |
| 6 | 2-Monoolein | 336 |
| 7 | 1-Oleoyl-2-palmitoylglycerol (OP) | 237 |
| 8 | 1-Palmitoyl-2-oleoylglycerol (PO) | 206 |
| 9 | 1,3-Dioleoyl-2-palmitoylglycerol (OPO) | 116 |
| 10 | 1,3-Dipalmitoyl-2-oleoylglycerol (POP) | 138 |

It was observed, from Table 2, that oleic acid, 2-monopalmitin, 1-monoolein and 2-monoolein increased the acylated ghrelin level about 1.5 to 2 times as compared with the control. Also, no differences of palmitic acid and 1-monopalmitin (10 μM) from the control were observed, but 1-monopalmitin (30 M) showed an increase in acylated ghrelin concentration as compared with the control.

It was observed, from Table 3, that oleic acid, 2-monopalmitin, 1-monoolein, 2-monoolein, 1-oleoyl-2-palmitoylglycerol (OP) and 1-palmitoyl-2-oleoylglycerol (PO) increased the acylated ghrelin concentration about 2 to 4 times as compared with the control. Also, it was observed that 1,3-dioleoyl-2-palmitoylglycerol (OPO) and 1,3-dipalmitoyl-2-oleoylglycerol (POP) increased the acylated ghrelin concentration as compared with the control.

It was observed, from FIG. 1, that 2-monopalmitin significantly increased the acylated ghrelin concentration about 1.2 times as compared with the control.

It was observed, from FIG. 2, that 2-monopalmitin, 1-monoolein and 2-monoolein significantly increased the acylated ghrelin concentration about 1.5 to 3 times as compared with the control.

As described above with respect to the results in Table 3, 1-oleoyl-2-palmitoylglycerol (OP), 1-palmitoyl-2-oleoylglycerol (PO), 1,3-dioleoyl-2-palmitoylglycerol (OPO), and 1,3-dipalmitoyl-2-oleoylglycerol (POP) were demonstrated to have a ghrelin secretion promoting effect. Meanwhile, this test system is in the absence of a gastric lipase. It is known that a triacylglycerol is physiologically hydrolyzed sn-3-position-specifically with a gastric lipase, producing a free fatty acid and a diacylglycerol in the stomach (ghrelin-secreting cells are present in the gastric mucosa), which is a site of action (Rogalska et al., JBC, 1990, 265, 33:20271-20276). Therefore, it was suggested that an acylglycerol (e.g., a triacylglycerol such as OPO or POP) producing 1-oleoyl-2-palmitoylglycerol (OP) or 1-palmitoyl-2-oleoylglycerol (PO) upon physiological hydrolysis with a gastric lipase also promote ghrelin secretion.

### Example 2: Example of Blending into Food Composition for Infants

A typical formulation (per 100 kcal) of the food composition for infants of the present invention is indicated in Table 4.

TABLE 4
Blending formulation of food composition for infants

| | | | Blending amount | | |
|---|---|---|---|---|---|
| Component | | | (per 100 kcal) | | |
| Protein | 2.20 | g | β-carotene | 1.4 | μg |
| Lipid | 5.16 | g | Vitamin B₁ | 0.079 | mg |
| Palmitic acid | 1.01 | g | Vitamin B₂ | 0.12 | mg |
| Palmitic acid bound to | 0.606 | g | Niacin | 0.59 | mg |
| β position | | | | | |
| Carbohydrate | 11.40 | g | Vitamin B₆ | 0.059 | mg |
| Ash | 0.45 | g | Vitamin B₁₂ | 0.40 | μg |
| α-Lactalbumin | 0.20 | g | Folic acid | 20 | μg |
| β-Lactoglobulin | 0.10-0.20 | g | Pantothenic acid | 0.85 | mg |
| Lactadherin | 4.0-9.9 | mg | Biotin | 0.51 | μg |
| Taurine | 5.5 | mg | Vitamin C | 14 | mg |
| Nucleotide | 2.8 | mg | Inositol | 18 | mg |
| Linoleic acid | 0.71 | g | Carnitine | 2.0 | mg |
| α-Linolenic acid | 0.085 | g | Choline | 12 | mg |
| Arachidonic acid | 13 | mg | Sodium | 28 | mg |
| Docosahexaenoic acid | 20 | mg | Potassium | 97 | mg |
| Cholesterol | 15 | mg | Chlorine | 61 | mg |
| Phospholipid | 50 | mg | Calcium | 75 | mg |
| Lactose | 10 | g | Magnesium | 7.9 | mg |
| Fructooligosaccharide | 0.40 | g | Phosphorus | 42 | mg |
| Vitamin A | 77 | μg | Iron | 1.2 | mg |
| Vitamin D | 1.3 | μg | Manganese | 7.9 | μg |
| Vitamin E | 1.2 | mg | Iodine | 0.01 | mg |
| Vitamin K | 4.9 | μg | Selenium | 2.1 | μg |

### Example 3: Example of Blending into Food Composition for Aged Persons

A typical formulation (per 100 ml) of the food composition for aged persons of the present invention is indicated in Table 5.

TABLE 5
Blending formulation of food composition for aged persons

| | | | Blending amount | | |
|---|---|---|---|---|---|
| Component | | | (per 100 ml) | | |
| Energy | 100 | kcal | Niacin | 2.4 | mg NE |
| Protein | 4.0 | g | Pantothenic acid | 0.60 | mg |
| Lipid | 2.8 | g | Vitamin K | 5.0 | μg |
| Palmitic acid | 0.6 | g | Choline | 1.7 | mg |
| Palmitic acid bound | 0.3 | g | Biotin | 15 | μg |
| to β position | | | | | |
| Carbohydrate | 15.5 | g | Sodium | 110 | mg |
| Saccharide | 14.5 | g | Potassium | 100 | mg |
| Dietary fiber | 1.0 | g | Chlorine | 140 | mg |
| Ash | 0.67 | g | Calcium | 60 | mg |
| Vitamin A | 60 | μg RAE | Phosphorus | 60 | mg |
| Vitamin D | 0.5 | μg | Magnesium | 20 | mg |
| Vitamin E | 3 | mg | Iron | 1.0 | mg |
| Vitamin C | 16 | mg | Manganese | 0.23 | mg |
| Vitamin B₁ | 0.15 | mg | Copper | 0.080 | mg |
| Vitamin B₂ | 0.20 | mg | Zinc | 0.80 | mg |
| Vitamin B₆ | 0.30 | mg | Chromium | 3.0 | μg |
| Vitamin B₁₂ | 0.60 | μg | Selenium | 3.5 | mg |
| Folic acid | 50 | μg | Iodine | 15 | μg |

*The “μg RAE” in this table denotes the retinol activity equivalent, and the “mg NE” denotes the niacin activity equivalent.

The provision of a method for producing a food comprising acylglycerol(s) and/or free fatty(s) acid which is/are highly safe leads to normal development of an appetite regulating function necessary for the target for feeding, including infants, to take necessary energy; functional development due to the physiological action of growth hormone; and prevention and amelioration of loss of appetite and reduction in energy intake of aged persons, as well as reductions in muscle mass and muscle strength due to reduced secretion of growth hormone, and thus can contribute to enhancement of nation's health.

1-10. (canceled)

11. A method for promoting ghrelin secretion, a method for promoting or enhancing appetite, a method for ameliorating or alleviating loss of appetite, a method for promoting or enhancing growth hormone secretion, a method for improving or alleviating malnutrition, a method for promoting or enhancing fat accumulation, a method for increasing body weight, a method for promoting or enhancing gastric acid secretion, or a method for promoting or enhancing gastric motility, the method comprising feeding or administering, to a subject in need thereof, one or more substance(s) selected from the group consisting of an acylglycerol comprising palmitic acid and/or oleic acid as a constituent fatty acid(s) and oleic acid, or a composition comprising the one or more substance(s).

12-13. (canceled)

14. The method according to claim 11, wherein, in the acylglycerol, the palmitic acid and/or the oleic acid are/is bound to sn-1 position and/or sn-2 position of glycerol.

15. The method according to claim 11, wherein the acylglycerol comprises the palmitic acid and the oleic acid as the constituent fatty acids.

16. The method according to claim 14, wherein, in the acylglycerol, an optional fatty acid is bound to sn-3 position of the glycerol.

17. The method according to claim 16, wherein the optional fatty acid is palmitic acid or oleic acid.

18. The method according to claim 11, wherein the acylglycerol is one or more selected from the group consisting of 1-monopalmitin, 2-monopalmitin, 1-monoolein, 2-monoolein, 1-oleoyl-2-palmitoylglycerol, 1-palmitoyl-2-oleoylglycerol, 1,3-dioleoyl-2-palmitoylglycerol, and 1,3-dipalmitoyl-2-oleoylglycerol.

19. The method according to claim 11, wherein the amount of the substance(s) in the composition is 1 to 95% by mass.

20. The method according to claim 11, wherein the composition is a food composition.

21. The method according to claim 11, the subject is infant and/or aged person.
